(19) United States
(12) Patent Application Publication
(10) Pub. No.: US 2022/0111616 A1
(43) Pub. Date: Apr. 14, 2022

(54) Elastic Laminate Having Increased Rugosity

(71) Applicant: Berry Global, Inc., Evansville, IN (US)

(72) Inventors: Iyad Muslet, West Chester, OH (US); Christopher A. Shelley, Lebanon, OH (US); Frank Eschenbacher, Schnelldorf (DE)

(73) Assignee: Berry Global, Inc., Evansville, IN (US)

(21) Appl. No.: 17/490,577

(22) Filed: Sep. 30, 2021

Related U.S. Application Data
(60) Provisional application No. 63/090,453, filed on Oct. 12, 2020.
(60) Provisional application No. 63/123,544, filed on Dec. 10, 2020.

(51) Int. Cl. B32B 7/05; B32B 5/02; B32B 25/10; B32B 25/18; B32B 25/14

(52) U.S. Cl. CPC B32B 7/05; B32B 5/022; B32B 25/10; B32B 2555/02; B32B 25/14; B32B 2307/718; B32B 2250/02; B32B 25/18

(57) ABSTRACT

Elastomeric laminates and methods of making elastomeric laminates comprising an elastomeric film point-bonded to an carded, spunbond or bicomponent nonwoven substrate to create bond sites, wherein each bond site is from about 3 mm to about 10 mm from each adjacent bond site, and further wherein the laminate comprises rugosities, optionally having a height of greater than 2 mm.

### RELATED APPLICATIONS

This application claims the benefit of priority of U.S. Provisional Patent Application No. 63/090,453, filed on Oct. 12, 2020 and of U.S. Provisional Patent Application No. 63/123,544, filed on Dec. 10, 2020, both of which are incorporated herein in their entirety.

### FIELD OF THE INVENTION

The present invention relates to elastomeric laminates comprising an elastomeric film and a nonwoven substrate, which exhibit increased loft and rugosity. The films and laminates are suitable for use in a variety of products, including single-use personal care products such as absorbent articles and face coverings.

### BACKGROUND

Consumers have indicated a preference for absorbent products, such as diapers and adult incontinence products, that utilize elastomeric laminates having increased rugosity. Also known as an “accordion look,” rugosity signals a high degree of stretchability and a soft feel. Such laminates also may allow increased air passage, which provides increased breathability.

Laminates having increased rugosity are known in the art, however, such laminates typically contain elastic strands. A number of disadvantages are associated with wearing products containing elastic strands in contact with the skin, including lines and red marks on the skin. In addition, stretching a laminate containing strands may cause the strands to bunch together, and the laminate becomes bulky. This, in turn, creates a more visible and a noisier product. A need exists, therefore, for elastomeric laminates that are sufficiently robust to withstand the manufacturing process and normal consumer use, that are soft and breathable, that are appealing to consumers, and that avoid the disadvantages associated with elastic strands.

### SUMMARY OF THE INVENTION

The present invention meets the aforementioned needs by providing elastomeric laminates comprising an elastomeric film and nonwoven, and which employ combination of a pattern of bonding which, when together with a suitable depth of intermeshing and post-lamination stretching, results in laminates having excellent strength, haptic, and aesthetic properties. The softness observed in these laminates typically is seen only in laminates comprising higher basis weight nonwovens. These properties are due in part to the laminates having significantly increased rugosity, or ridges, which far exceed the rugosity observed in comparative laminates that are not bonded and stretched as described herein.

In one aspect, the present invention provides methods of making an elastomeric laminate comprising the steps of joining an elastomeric film to at least one nonwoven substrate by means of point-bonding to create bond sites; and, stretching the laminate by means of cross-directional intermeshing to create an intermeshed laminate, wherein the intermeshed laminate comprises rugosities, and wherein each bond site is from about 3 mm to about 10 mm from each adjacent bond site.

In another aspect, the present invention provides elastic laminates comprising an elastomeric film point-bonded to a carded, spunbond or bicomponent nonwoven substrate to create bond sites, wherein each bond site is from about 3 mm to about 10 mm from each adjacent bond site, and further wherein the laminate comprises rugosities having a height of greater than 2 mm.

In yet another embodiment, the present invention provides articles, for example single-use absorbent articles, comprising the laminates described herein.

### BRIEF DESCRIPTION OF THE DRAWINGS

FIG. 1 is a photo of a laminate comprising a 35 gsm elastic film between two 24 gsm carded nonwovens. The laminate was stretched by CD-intermeshing at a depth of 0.08″, and displays the desired rugosity and loft.

FIG. 2 is a photo of a laminate comprising a 35 gsm elastic film between two 24 gsm carded nonwovens. The laminate was stretched by CD-intermeshing at a depth of 0.12″, and displays the desired rugosity and loft.

FIG. 3 is a photo of a laminate comprising a 35 gsm elastic film between two 24 gsm carded nonwovens. The laminate was stretched by CD-intermeshing at a depth of 0.14″, and displays the desired rugosity and loft.

FIG. 4 is a photo of a laminate comprising a 35 gsm elastic film between two 24 gsm carded nonwovens. The laminate was stretched by CD-intermeshing at a depth of 0.14″ and the bond points were aligned with the CD-intermeshing stripes. The laminate displays the desired rugosity and loft.

FIG. 5 is a photo of a comparative laminate comprising a 35 gsm elastic film between two 20 gsm spun-bond nonwovens, wherein the distance between the bonds is less than 3 mm. The laminate does not display the claimed rugosity.

FIG. 6 is a photo of a comparative laminate comprising a 35 gsm elastic film between two 24 gsm carded nonwovens, wherein the laminate was not subjected to intermeshing after lamination. The laminate does not display the claimed rugosity.

FIGS. 7(a) and (b) depict two non-limiting examples of bond patterns which, when stretched, result in the laminates of the present invention.

FIG. 8 depicts measurement and calculation of rugosity in laminates comprising a single nonwoven (bilaminate).

### DETAILED DESCRIPTION OF THE INVENTION

“Accordion-like appearance” means that a laminate comprises rugosities that, when the material is stretched, substantially flatten, and which return when the stretching force is removed.

“Bond distance,” “distance between bonds,” or variants thereof, means the shortest distance measured between the outer circumferences of two adjacent bonds. When the bonds are the result of ultrasonic bonding, the bond distance is measured on the anvil, which houses the bond pattern. Alternatively, the bond distance may be measured on the finished laminate while the laminate is retained in a maximally stretched position.

“Elastomeric,” “elastomer,” “elastic,” or variants thereof, mean a film or a laminate that, when stretched beyond the original length, recovers to no more than about 1.2 times the original length in the direction of the applied stretching force.

“Elastic stretch,” “elasticity,” or variants thereof, means the degree to which a film or laminate may be stretched before breaking or becoming permanently deformed. Elastic stretch is typically expressed as a percentage of the original length. For example, an elastic stretch of 100% means that a film or laminate may be stretched to about twice its original length before breaking or permanent deformation.

“Gsm” means grams per square meter, and is a measure of the basis weight, which is an industry standard term that quantifies the thickness or unit mass of a film or laminate product.

“Load peak,” as used herein, means the maximum tensile force that can be applied before permanent deformation.

“Permanent set” is the permanent deformation of a material after removal of an applied load. In the case of elastomeric films or laminates, permanent set is the increase in length of a sample after the film has been stretched to a given length and then allowed to relax as described herein. Permanent set is typically expressed as a percent increase relative to the original size, therefore, a film that recovers to a length of 1.2 times the original length is said to have a permanent set of about 20%.

“Polyethylene rich,” or alternatively, “polyethylene-based,” means a polymeric composition comprising at least about 60% by weight of polyethylene monomers. “Polyethylene rich” or “polyethylene-based” is understood not to include polymers comprising mixtures of ethylene and propylene monomers, such as poly(ethylene-propylene).

“Polypropylene rich,” or alternatively, “polypropylene-based,” means a polymeric composition comprising at least about 60% by weight of polypropylene monomers. “Polypropylene rich” or “polypropylene-based” is understood not to include polymers comprising mixtures of ethylene and propylene monomers, such as poly(ethylene-propylene).

“Pre-activation,” or “activation,” or any variants thereof, mean a process by which the elastomeric film or other material is rendered more easily stretchable, for example by stretching and allowing the material to relax prior to lamination.

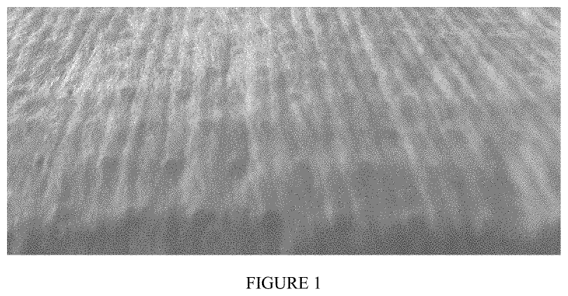

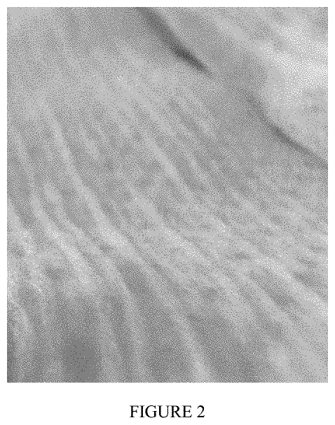

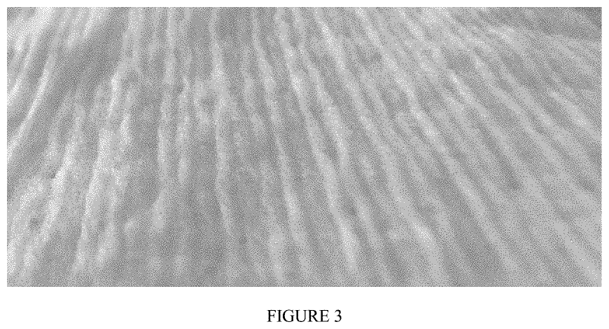

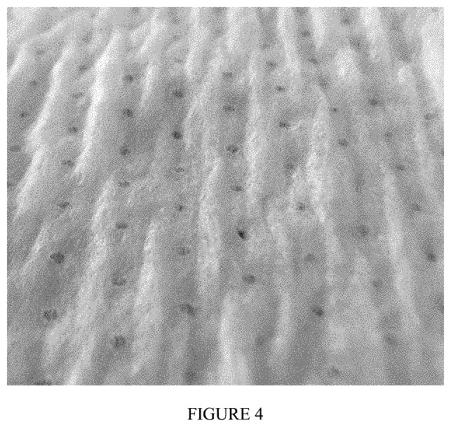

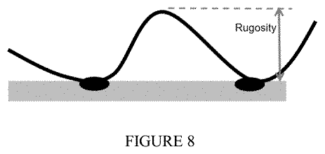

“Rugosity,” as used herein, means the average height of ridges of stretched, non-bonded substrate (typically a nonwoven) which extend above the plane of the film in a laminate comprising a single nonwoven, or below the plane of the film in a laminate comprising two substrates, measured as shown in FIGS. 8. A laminate has suitable rugosity if average height of the ridges is at least 2 mm+/−1 mm, as shown in FIGS. 1-4.

“Non-stranded” means that the laminate is substantially free of elastic strands.

The laminates of the present invention comprise one or more elastomeric films and one or more nonwovens. Whereas a variety of elastomeric films may be suitable for use, particularly desirable films include elastomeric films comprising one or more styrenic block copolymers as described herein. The films further may comprise polystyrene. When subjected to testing by the tensile test, the films, and laminates comprising the films, exhibit an elastic stretch of at least 60% and/or a permanent set of less than 25%.

The films are coextruded multilayer films and may have a structure in which relatively elastomeric layers (B) are alternated with relatively inelastic layers (A). In one particular embodiment, the films have a structure denoted by ABA, wherein A is the outer, or skin, layer and B is the inner, or core, layer. However, variations in the number and arrangement of the layers would be readily apparent to one of skill in the art.

The core layer (or layers in a film having more than three layers) may comprise one or more styrenic block copolymers (SBCs) , olefinic block copolymers (OBCs), or random block copolymers, including styrene-butadiene-styrene (SBS), styrene-isoprene-styrene (SIS), styrene-isoprene-butylene-dtyrene (SIBS), styrene-ethylene-butylene-styrene (SEBS), styrene-ethylene-propylene (SEP), styrene-ethylene-propylene-styrene (SEPS), or styrene-ethylene-ethylene-propylene-styrene (SEEPS) block copolymer elastomers, and copolymers and mixtures of any of the foregoing. Although any SBC may be used, particularly useful SBCs in the films of the present invention are non-hydrogenated SBCs, including but not limited to SBS, SIS and SIBS. Non-limiting examples of SBCs suitable for use in the present invention include those available from Dexco Polymers, Plaquemine, La., for example, VECTOR® 4111A and 7620.

Olefinic block copolymers suitable for use in the core layer include polypropylene-based (also termed “propylene-rich”) olefinic block copolymers (OBCs) such as those sold under the trade name INFUSE®, including INFUSE 9507 and 9100, by The Dow Chemical Company of Midland, Mich. Other suitable copolymers include the trade names VISTAMAXX® and IMPACT®, for example VISTAMAXX 6102, available from ExxonMobil Chemical Company of Houston, Tex. In one embodiment, OBCs suitable for use in the present invention have a density of from about 0.85 to about 0.89 grams per cubic centimeter (g/cm3).

The total amount of SBCs in the core layer may be at least about 50%, from about 50% to about 99%, from about 60% to about 99%, from about 50% to about 95%, from about 55% to about 95%, from about 60% to about 95%, from about 65% to about 95%, from about 70% to about 95%, from about 75% to about 95%, from about 80% to about 95%, from about 70% to about 90%, or alternatively from about 80% to about 90%.

The core layer further may comprise polystyrene in an amount of about 30% or less, and alternatively 25% or less, 20% or less, or from about 1% to about 30%, from about 5% to about 25%, or from about 5% to about 20%. One example of polystyrene suitable for use in the present invention is STYROLUTION 3190, available from PolyOne Corporation, Avon Lake, Ohio.

The films further may comprise other elastomeric polymers, such as elastomeric olefinic random copolymers, polyurethanes, rubbers, vinyl arylenes and conjugated dienes, polyesters, polyamides, polyethers, polyisoprenes, polyneoprenes, copolymers of any of the above, and mixtures thereof.

The outer layers (the A-layers, or skin layers) each may comprise polypropylene in an amount of at least 10%, at least 15%, at least 20%, at least 25%, from about 1% to about 90%, from about 1% to about 85%, from about 1% to about 80%, or from about 1% to about 75%. In one embodiment, the polypropylene is present in an amount of at least 20%, and in another embodiment is present in an amount of from about 20% to about 85%.

Each outer layer further may comprise about 2.5%, 5%, 7.5%, 10%, 15% or 20% of the total film thickness. In some embodiments, the outer layers further each may have a thickness of from about 1% to about 20%, from 3% to about 15%, or from about 5% to about 15% of the total thickness of the film. Alternatively, the outer layers each may have a thickness of from about 1 micron to about 20 microns, or from about 1 microns to about 15 microns, from 1 micron to about 10 microns, from about 1 microns to about 7 microns, and alternatively from about 1 microns to about 5 microns. By way of illustration only, if the total thickness of the film is 100 microns and each outer layer has a thickness of 5 microns, then the outer layers comprise a total of 10% of the film thickness.

The polypropylene in the outer layers may comprise polypropylene, homopolymer polypropylene, impact copolymer polypropylene, as well as other types of polypropylene that would be apparent to one of skill in the art.

The films further may comprise a filler suitable to induce pore formation upon stretching, including but not limited to calcium carbonate. The films may include master batch and optional components or fillers, such as opacifiers, plasticizers, compatibilizers, draw down polymers, processing aids, anti-blocking agents, viscosity-reducing polymers, and the like.

The films may have a basis weight of 100 gsm or less, 60 gsm or less, 50 gsm or less, 45 gsm or less, 40 gsm or less, 35 gsm or less, 30 gsm or less, 25 gsm or less, 20 gsm or less, and alternatively from about 5 gsm to about 50 gsm, including any and all sub-ranges encompassed therein, including, by way of example only, from about 10 gsm to about 40 gsm or from about 25 gsm to about 75 gsm.

The film may be unactivated or activated prior to being bonded to the nonwoven substrate (i.e., “preactivated”). Activation may occur offline, as part of a process separate from the lamination process, or inline with the lamination process.

The laminates of the present invention comprise a substrate attached to one or both surfaces of the film. Whereas a variety of substrates may be useful, the substrates of the present invention are nonwoven substrates. The laminates may comprise more than one film and more than one substrate. For example, the laminate may be a bilaminate, comprising one film and one nonwoven; a trilaminate comprising one film between two nonwovens; or a multi-laminate, having a variety of structures, including two films sandwiched between three nonwovens, or two bilaminates joined at the surface of each film.

Each substrate is attached to a film by means of point-bonding. A variety of means of point-bonding may be employed, including ultrasonic bonding, adhesive lamination, extrusion lamination, calendaring, as well as other means that would be known to one of skill in the art. In one embodiment, the laminate is ultrasonically bonded, with the resulting laminates comprising ultrasonic welds, or bonds.

Suitable substrates that produce the desired rugosity when bonded in the manner described herein include nonwovens comprising bicomponent fibers comprising polyethylene and polypropylene in a core/sheath structure (“bico” nonwovens), carded nonwovens, and spunbond nonwovens, including those comprising a blend of polyethylene and polypropylene, one example of which are those sold under the tradename SOF SPAN (available from Berry Global, Inc.). The nonwoven substrate may have a basis weight of about 100 gsm or less, alternatively about 50 gsm or less, alternatively about 25 gsm or less, alternatively about 20 gsm or less, and alternatively from about 5 gsm to about 100 gsm, including any and all sub-ranges encompassed therein, for example, from about 5 gsm to about 50 gsm, 10 gsm to about 25 gsm and from about 15 gsm to about 20 gsm.

The nonwoven may be extensible, meaning that the nonwoven is capable of being stretched by e.g. intermeshing or SELFing or other suitable means of stretching. Upon stretching, breakage of fibers may occur, however the nonwoven substantially maintains its form. An extensible nonwoven may or may not be elastomeric, meaning that an extensible nonwoven may not contract after stretching as would an elastomeric nonwoven. The nonwovens further are non-stranded. The nonwoven may have a load peak of less than 6 N/cm and/or an elastic stretch that is more that 50%. The laminates may have a permanent set of less than 25%, less than 20%, less than 15%, less than 10%, or less than 5%. In addition, the laminates may have a load at break of at least 100%, meaning that the laminate may be stretched to at least twice its original length before breaking.

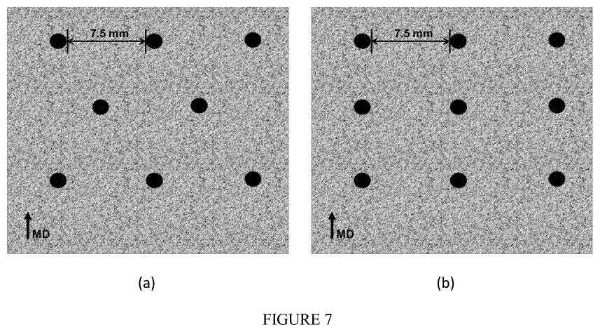

The laminates of the present invention comprise a point-bonded pattern in which each individual bond is sufficiently distanced from each adjacent bond to result in the desired rugosity upon stretching. Non-limiting examples of suitable bond patterns are depicted in FIGS. 7(a) and (b). As would be understood by one of skill in the art, other patterns, for example, patterns comprising wavy lines, geometric shapes, etc., may be used to achieve the desired rugosity. In one embodiment, the bonds may comprise parallel lines of bonds, or rows, to create a square pattern (FIG. 7(a)), or parallel lines, or rows, of offset bonds to create a diamond-shaped pattern (FIG. 7(b)). In one embodiment, the lines of bond sites are aligned with the space between CD-intermeshing gears, meaning that the bond sites fall within the “intermeshing stripes” that result from the space between the intermeshing gears. It has been found that a distance between each of the individual bonds of at least about 3 mm, in each direction, as measured as described herein, is necessary to achieve the desired rugosity. In one alternative embodiment, the distance between adjacent bonds after lamination is from about 3 mm to about 10 mm from one another.

In one embodiment, the point-bonds are substantially circular. Alternatively, the point-bonds may be oval-shaped or irregularly-shaped. In yet another embodiment, the point-bonds are elongated to resemble a short line. In one embodiment, the elongated point-bonds may have a length of from about 1 to 5 mm.

### Method

An apparatus and methods suitable for making the films used in the laminates is described in, e.g., U.S. Pat. No. 7,442,332 (Cancio et al.). The films may be coextruded, and may be cast, blown, or formed by any other method that results in the films described herein. In one embodiment, the films are blown films. As stated previously, a variety of means may be used to laminate the film to the substrate. The film may be laminated by ultrasonic point-bonding, using an apparatus described, e.g., in U.S. Pat. No. 9,498,941 (Sablone et al.) with a patterned anvil roll. The film may be extrusion laminated by using a patterned chill roll, or calendared by using a patterned calendar roll, to impart a suitable pattern. Alternatively, the film may be adhesively bonded to the substrate.

In one embodiment, the laminates comprise a film and a nonwoven substrate, which are lightly extrusion-bonded using controlled compression to hold the film and nonwoven substrate together long enough to be point-bonded or ultrasonically-bonded.

Upon lamination, the resulting laminates are stretched by intermeshing, for example, by cross-direction (CD) intermeshing or alternatively by SELFing, at a depth which is sufficient to produce the desired rugosity and loft. CD intermeshing may be performed at a depth of from about 0.08 inches to about 0.200 inches, and alternatively at a depth of from about 0.100 inches to about 0.160 inches.

The rugosities of the laminates of the present invention have an average height of at at least about 1 mm, and in alternative embodiments the average height is at least 2 mm, at least 3 mm, at least 4 mm, at least 5 mm, at least 6 mm, at least 7 mm, at least 8 mm, at least 9 mm, and at least 10 mm. In an alternative embodiment, the average height of the rugosities is from about 2 to about 10 mm.

The films and/or laminates of the present invention may be incorporated into a variety of products, including, for example, single-use absorbent products such as diapers, training pants, adult incontinence pads and pants and swimwear.

The films and/or laminates are particularly useful as fasteners, waistbands and leg cuffs of absorbent articles. Accordingly, in one embodiment, the present invention is related to an absorbent article comprising the films and/or laminates described herein. In one embodiment, the absorbent article is a diaper. Other uses include as diaper backsheets or ears (closure tabs), pouches for packaging, and wrapping products for such as personal hygiene items. In another embodiment, the laminates of the present invention may be used in gloves, face masks, or other types of apparel.

### Test Methods

Permanent set is measured by the Two Cycle Hysteresis Test, as described in U.S. Patent Publication 2016/0200080 (Muslet et al.), with the following changes: All specified percent engineering strains are 100% as opposed to 130%. The percent set is defined as the percent engineering strain after the start of the second load cycle (from segment 6) where a force of 10 grams is measured (percent set load=10 grams). The temperature of the room is about 73° F.+/−5° F. (i.e., engineering strain=130%).

“Tensile strength,” means the load required to induce a break (“load at break”) in the film in either the cross-direction (CD) or the machine-direction (MD). Tensile strength is expressed in units of N/cm or equivalent units thereof, and is determined by ASTM method D822-02, using the following parameters: Sample Direction=MD×CD; Sample size=1 inch width×6 inch length; Test speed=20 in/min; Grip distance=2 inch. Grip size=3 inch wide rubber faced grips evenly gripping sample.

The rugosity is measured as follows: A laminate is laid flat and secured without stretching. In a given rugosity wherein the nonwoven is not attached to the underlying film, the rugosity is “tented” to its maximum height with the aid of a pin or other suitable object. The height in mm of the apex of the rugosity from the surface of the underlying film is measured with a ruler.

### EXAMPLE 1

The laminates comprised elastomeric films having a basis weight of 30-35 gsm and made of three layers (A/B/A construction). The outer A-layers (skins) are made of blend of polypropylene and polyethylene. The inner B-layer is made blend of SIS (80%), polystyrene (15%), with the remainder comprising white master batch and process aid master batch. The film is co-extruded and then activated using in-line CD-intermeshing gears. The film is sandwiched between two layers of carded or spunbond nonwovens, each having a basis weight of approximately 22 gsm. The laminate (NW/Film/NW) is then ultrasonically point bonded using an ultrasonic laminator that has one anvil and ultrasonic horns that line the entire width of the anvil.

When the laminate is passed between the anvil and the horn, bond points are created and the three materials are welded together at these points. This process results in a relatively flat laminate. The laminate is then stretched by being passed through intermeshing gears. During the activation the film and the nonwovens which are attached at the point welds are stretched. After activation, the laminate is allowed to relax. The distance after lamination between any two points is about 5-7 mm. The film retracts, however, the nonwovens remain extended. In the space between the point-bonds, where the nonwovens are not attached to the film, the nonwovens bulge and extend perpendicularly away from the film (i.e., above and below the plane of the laminate). Therefore, when the elastic film retracts, the nonwovens corrugate and result in the desired rugosity.

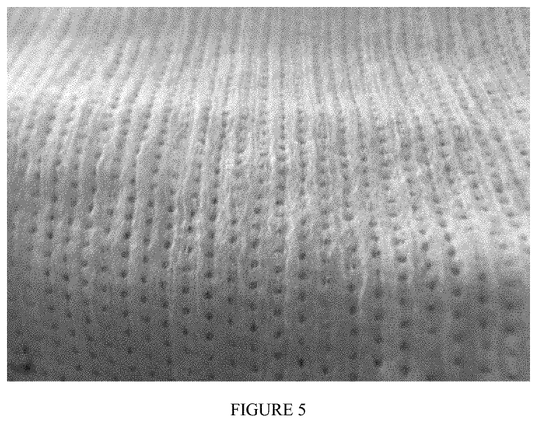

It was found that when the distance between adjacent point-bonds is less than 3 mm (about 2.5 mm, as shown in FIG. 5), the laminate did not exhibit the claimed rugosity or good stretchability. Furthermore, the laminate exhibited tearing during activation due to the tension created by the shortened distance between the bonds.

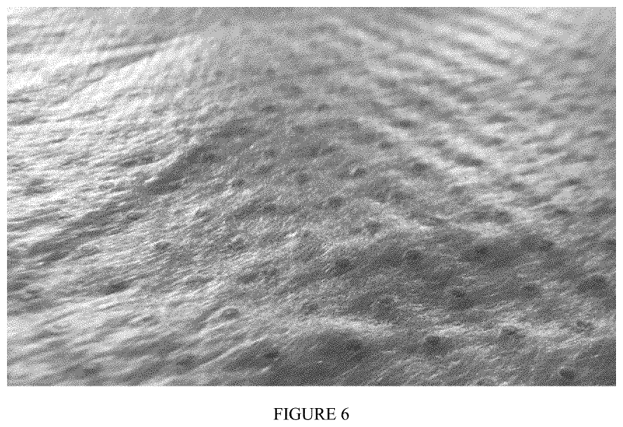

As can be seen in FIGS. 1-4, the laminates produced by the above process exhibit a rugosity of at least 2 mm (i.e., the “accordion look”) and have excellent aesthetic and haptic qualities. In contrast, the laminate shown in FIG. 5, in which the distance between bonds is less than 3 mm, exhibits limited rugosity of less than 2 mm and unacceptable haptic properties. Similarly, the laminate shown in FIG. 6, which was not subjected to intermeshing after lamination, exhibits essentially no rugosity (less than 1 mm) and undesirable haptic properties.

### EXAMPLE 2

Laminates comprising two 20 gsm nonwovens (SOFSPAN, available from Berry Global) and a blown 30 gsm film having an A/B/A construction. The film is pre-activated off-line. The A-layers comprise 55% LLDPE, 10% homo-PP, and 12% LDPE, with the remainder comprising master batch and processing aids. The B-layer comprises 98% VISTAMAXX 6102, with the remainder comprising processing aids. The film and nonwovens are laminated by ultrasonic bonding as in Example 1, followed by CD-intermeshing at a depth of 3.0 mm.

All documents cited in the Detailed Description of the Invention are, in relevant part, incorporated herein by reference; the citation of any document is not to be construed as an admission that it is prior art with respect to the present invention. To the extent that any meaning or definition of a term in this document conflicts with any meaning or definition of the same term in a document incorporated by reference, the meaning or definition assigned to that term in this document shall govern. All ranges are inclusive and combinable. To the extent a value is not explicitly listed, it is understood to be implied as an option if included in a recited range.

Whereas particular embodiments of the present invention have been illustrated and described, it would be obvious to those skilled in the art that various other changes and modifications can be made without departing from the spirit and scope of the invention. It is therefore intended to cover in the present claims all such changes and modifications that are within the scope of this invention.

What is claimed is:

1. A method of making a non-stranded elastomeric laminate comprising the steps of joining an elastomeric film to at least one nonwoven substrate by means of point-bonding to create bond sites having a distance of from about 3 mm to about 10 mm from each adjacent bond site; and, stretching the laminate by means of cross-directional intermeshing to create an intermeshed laminate comprising rugosities.

2. The method of claim 1, wherein the rugosities have an average height of greater than 2 mm.

3. The method of claim 1, wherein the cross-directional intermeshing is performed at a depth of from about 0.08 inches to about 0.2 inches.

4. The method of claim 1, wherein the nonwoven substrate is a carded, spunbond, or bicomponent nonwoven.

5. The method of claim 1, wherein the point bonding is performed by ultrasonic bonding.

6. The method of claim 1, wherein the bond sites are arranged in rows or in offset rows.

7. The method of claim 6, wherein at least one offset row is aligned with the cross-directional intermeshing.

8. The method of claim 1, wherein the elastomeric film comprises a SIS, SBS, SEBS, an olefinic block copolymer, a random block copolymer, or combinations thereof.

9. The method of claim 1, wherein the film is a blown film.

10. The method of claim 1, wherein the basis weight of the elastomeric film is less than about 60 gsm.

11. The method of claim 1, wherein the basis weight of the nonwoven substrate is less than about 100 gsm.

12. A method of making a non-stranded elastomeric laminate comprising the steps of joining an elastomeric film to at least one nonwoven substrate by means of point-bonding to create bond sites having a distance of from about 3 mm to about 10 mm from each adjacent bond site; and, stretching the laminate by means of cross-directional intermeshing to create an intermeshed laminate comprising rugosities.

13. A method of making a non-stranded elastomeric laminate comprising the steps of joining a blown elastomeric film to at least one nonwoven substrate by means of point-bonding, to create bond sites having a distance of from about 3 mm to about 10 mm from each adjacent bond site; and, stretching the laminate by means of cross-directional intermeshing at a depth of from about 0.08 inches to about 0.2 inches to create an intermeshed laminate comprising rugosities.

14. A non-stranded elastic laminate comprising an elastomeric film point-bonded to an extensible carded, spunbond or bicomponent nonwoven substrate to create bond sites, wherein each bond site is from about 3 mm to about 10 mm from each adjacent bond site, and further wherein the laminate comprises rugosities having a height of greater than 2 mm.

15. The laminate of claim 14, wherein elastomeric film comprises SIS, SBS, SIBS, or combinations thereof.

16. The laminate of claim 14, wherein the elastomeric film comprises an olefinic block copolymer, a random block copolymer, or combinations thereof.

17. The laminate of claim 14, wherein the core layer comprises a polypropylene-based olefinic block copolymer, a random block copolymer, or combinations thereof.

18. The laminate of claim 14, wherein the elastomeric film has a basis weight of about 60 gsm or less.

19. The laminate of claim 14, wherein the laminate has a permanent set of less than about 25%.

20. The laminate of claim 14, wherein the bond sites are arranged in rows or in offset rows.
